(19) United States
(12) Patent Application Publication
(10) Pub. No.: US 2014/0010900 A1
(43) Pub. Date: Jan. 9, 2014

(54) PHARMACEUTICAL COMPOSITION CONTAINING ACTIVE VEGETABLE SUBSTANCES

(71) Applicant: Marco Spitz, Binzen (DE)

(72) Inventor: Marco Spitz, Binzen (DE)

(73) Assignees: MAX ZELLER SÖHNE AG, Romanshorn (CH); MEPHA SCHWEIZ AG, Aesch (CH)

(21) Appl. No.: 13/996,215

(22) PCT Filed: Dec. 21, 2011
(86) PCT No.: PCT/EP2011/073499

§ 371 Date: Sep. 26, 2013

(30) Foreign Application Priority Data
Dec. 21, 2010 (CH) 02142/10

(51) Int. Cl. A61K 47/38; A61K 36/16; A61K 47/32; A61K 36/38

(52) U.S. Cl. CPC A61K 47/38; A61K 36/38; A61K 36/16; A61K 47/32
USPC 424/730; 424/752

(57) ABSTRACT

The invention relates to a pharmaceutical composition containing active vegetable substances and a mixture made of a polymeric water-soluble and a polymeric water-insoluble matrix forming agent. Said pharmaceutical composition is suitable for the oral administration of active vegetable substances with delayed release.

The present invention relates to a pharmaceutical composition containing active plant substances in solid form for the oral administration of active plant substances with delayed release.

One option for decelerating the release of a substance from a solid pharmaceutical composition provides for embedding the active substance in a polymer matrix. The delay or deceleration, respectively, of the release of the active substance depends of the selection of the matrix-forming agent, additional excipients, the solubility of the active substance and the percentages that the individual components represent in a solid pharmaceutical composition. Generally, the rate of release of an active substance that is embedded in a matrix, for example in form of a tablet, is constant; meaning, it is linear.

Known medicinal products with a polymermatrix comprising incorporated active substances, typically contain synthetically prepared, uniform chemical compounds as active substances. However, orally administered phytomedicinal products with delayed release are not known in the art. From a galenic perspective, the decelerated release of pharmacologically active substances from plants, plant components, plant extracts and oils (hereafter referred to as active plant substances) from solid pharmaceutical compositions is a special challenge, because plant-based medicinal products represent a blend of different active substances with different solubilities. Although plant preparations with immediate release result in fast efficacy, said efficacy only has a limited duration. For patients, it is therefore desirable to extend the efficacy over a longer period of time.

Correspondingly, it is the object of the present invention to provide an orally administered pharmaceutical composition in solid form of active plant substances with delayed release, releasing the desired pharmacologically active components at a constant rate, or almost constant rate. Advantageously, at least 80% of the active plant substances has been released after 6-12 hours.

The invention relates to a pharmaceutical composition in solid form for the oral administration of active plant substances having a delayed release and comprising a plant preparation that contains the pharmacologically efficacious substances and a mixture of water-insoluble and water-insoluble polymeric matrix-forming agents.

Considered as solid forms for the oral administration are tablets, mini-tablets, pellets, coated tablets, capsules, for example soft gelatin capsules, and related forms, particularly tablets, mini-tablets and pellets, preferably tablets.

Considered as plant components are, for example, roots (such as Gentianae radix), rhizomes, bark (such as willow bark), flowers (such as melissa flowers), leaves (such as stinging nettle leaves), seeds (such as Indian psyllium husks, flaxseeds) and leaves with blossoms.

Considered as plant extracts and extract combinations are hypericum extract (St. John's wort), Ginkgo biloba extract, grape seed extract, passionflower extract, birchtree leaves extract, stinging nettle root extract, ivy extract, silver thistle extract, Devil's claw extract, echinacea extract, Gentianae radix extract, Sabalisserrulatae fructus extract, hawthorn extract, melissa leaf extract, hamamelis leaf extract, haronga bark extract, agnus castus extract, cimicifugaracemosa extract, valerian root extract, hop extract, petasites hybridus(butterbur) extract, horse-chestnut extract, bugleweed extract and combinations thereof, preferably hypericum extract and Ginkgo biloba extract.

Considered as plant oils are, for example, evening primrose oil, borage seed oil, menthol oil and chamomile oil.

The preferred solution is a pharmaceutical composition according to the invention that contains a plant extract, particularly hypericum extract. Also preferred is a pharmaceutical composition containing a Ginkgo biloba extract.

If a water-insoluble matrix-forming agent is used, almost uniform delayed release is observed. Depending on the selection of the water-insoluble matrix-forming agent, however, it can be difficult to achieve the release of the entirety of the active substance, or almost the entirety of the active substance, from the solid pharmaceutical preparation. If a water-soluble matrix-forming agent is used, the release is complete; however, the rate of the delayed release relative to a solid pharmaceutical composition without matrix-forming agent is not very high, even if the content % of the matrix-forming agent in the pharmaceutical composition is 50% or more.

Therefore, according to the invention, polymeric matrix-forming agents are used that area mixture of water-soluble and water-insoluble polymeric matrix-forming agents.

Polymeric water-soluble matrix-forming agents are, for example, hydroxypropyl methylcellulose (Hypromellose, HPMC), hydroxypropylcellulose (HPC), poloxamers, polyvinylpyrrolidone (povidone) and the like. HPMC is a mixture of alkyl-substituted cellulose with different polymerization levels and different substitution levels by methyl groups and 2-hydroxypropyl groups; it is commercially available in different viscosities, for example Methocel™ by the company Colorcon. Poloxamers are block copolymers from ethylene oxide and propylene oxide and known in the art as pharmaceutically usable matrix-forming agents under the trade name Lutrol™.

Considered as polymeric water-insoluble matrix-forming agents are, for example, ethylcellulose (for example, available under the trade name Ethocel™ by the company Colorcon), vinylpyrrolidone/vinylacetate copolymers or poylvinylpyrrolidone/polyvinylacetate mixtures (for example Kollidon SR™), polyacrylic acid copolymers, (for example, Carbopol™, polymers from acrylic acid with polyalkenylether, for example with allyl-pentaerthrit, or with divinylglycol) or polymethacrylic acid and/or methacrylic acid ester copolymers, for example Eudragit™ NM 30 D. The named water-insoluble polymers and copolymers are typically used as film-forming agents for coating tablets, capsules and the like.

Using mixtures of the aforementioned polymeric water-soluble and water-insoluble matrix-forming agents, it is possible to decelerate the active substance release, while, surprisingly, the active substance is completely released from the solid pharmaceutical compositions. The desired release rate can be influenced by suitable mixing ratios, wherein, simultaneously, it can be ensured that the release is linear or almost linear. In such mixtures, the ratio of water-soluble to water-insoluble matrix-forming agents is between 15:85 and 85:15, preferably between 25:75 and 75:25, especially preferred between 35:65 and 65:35.

Especially preferred pharmaceutical compositions contain a plant-based content that is over 50%, preferably above 60%, and especially preferred above 65%, with the matrix-forming agent or the mixture of matrix-forming agents at a content share of 15 to 50%, preferably 25 to 35%, especially preferred ca. 30%.

The pharmaceutical compositions according to the invention contain excipients, if necessary, for example, the usual carrier substances for solid oral administration forms such as, for example, microcrystalline cellulose, silicon dioxide, xanthan, magnesium aluminumsilicate, calcium silicate, calcium phosphate, magnesium phosphate, aluminum oxide or titanium dioxide; thinning agents such as, for example, calcium carbonate, calcium sulfate, hydrogenated vegetable oil, kaolin, magnesium carbonate, tribasic calcium phosphate, talc or sodium chloride; gliding agents such as, for example, colloidal silicon dioxide, starch or talc; mold release agents such as, for example, calcium stearate, zinc stearate, magnesium stearate, stearinic acid, fumaric acid, glycerin monostearate, glycerol palmitostearate, mineral oil, sodium benzoate, sodium lauryl sulfate, sodium stearyl fumarate, talc, hardened castor oil or hydrogenated castor oil; color pigments such as, for example, titanium dioxide, iron oxides, lacquered synthetic indigo or lacquered erythrosin; sugars such as, for example, mannite, sorbite, dextrins, maltodextrins, inosite, isomalt, lactite, maltite and xylite; and other flavoring agents such as, for example, aroma agents or artificial sweeteners such as, for example, acesulfame, aspartame, cyclamate, saccharin, sucralose orthaumatin. Further considered excipients are polyhydroxy compounds, for example ethylene glycol, propylene glycol or butylene glycol, diethylene glycol, triethylene glycol, tetraethylene glycol and polyethylene glycol, glycerin or glycerin derivatives partially etherized with ethylene oxide.

The pharmaceutical compositions according to the invention can also contain excipients that, owing to their good solubility, form pores in the polymeric matrix. Considered as “pore-forming agents,” materials of this kind are typically water-soluble substances such as, for example, sugar, for example, mannite, sorbite, isomalt, glucose, saccharose, maltite and erythrite.

By combining a water-soluble and a water-insoluble matrix-forming agent, if necessary in connection with a pore-forming agent and further tableting excipients, it is possible to achieve any desired delayed release effect in highly dosed preparations of plant extracts, such as hypericum with an extract content in excess of 50% in the tablet, while adjusting, simultaneously, a preferred release profile.

The invention will be explained in further detail based on the examples below that are in no way intended to limit the scope of protection.

### COMPARISON EXAMPLE 1

### Preparation of a Composition According to the Invention Containing Hypericum Extract and Kollidon® SR, a Water-Insoluble Polymeric Matrix-Forming Agent

### Composition

| | Batch | Quantity per | |
|---|---|---|---|
| Substance | quantity [g] | tablet [mg] | Content % |
| Hypericum extract | 750.00 | 500.00 | 69.44% |
| Kollidon ® SR | 283.05 | 188.70 | 26.21% |
| (=polyvinylacetate/ | | | |
| polyvinylpyrrolidone blend) | | | |
| Aerosil ®200 (silicon dioxide) | 10.80 | 7.20 | 1.00% |
| Talc | 30.75 | 20.50 | 2.85% |
| Magnesium stearate | 5.40 | 3.60 | 0.50% |
| Total | 1080.00 | 720.00 | 100.00% |

### Preparation

The hypericum extract is mixed with Kollidon® SR, Aerosil® 200 and talc for 10 minutes in a tumbling mixer. After adding magnesium stearate, mixing is continued for another 5 minutes.

Using a model KilianSP300 eccentric press, the finished mixture is pressed into oval tablets with a breaking strength of 200 N.

### Active Substance Release

The active substance release from the tablet was established in a buffer of pH 7.2 over a period of 10 hours. The released amount of hypericin was measured.

| | Active substance |
|---|---|
| Time [h] | release [%] |
| 0 | 0 |
| 1 | 19.5 |
| 2 | 28.0 |
| 3 | 34.2 |
| 4 | 40.4 |
| 5 | 42.9 |
| 6 | 45.1 |
| 7 | 45.9 |
| 8 | 46.5 |
| 9 | 46.8 |
| 10 | 47.1 |

As the table shows, the active substance has been released only inadequately even after 10 hours (<50%).

### COMPARISON EXAMPLE 2

Preparation of a composition according to the invention containing hypericum extract and Carbopol, a water-insoluble polymeric matrix-forming agent.

### Composition

| | Batch | Quantity per | |
|---|---|---|---|
| Substance | quantity [g] | tablet [mg] | Content % |
| Hypericum extract | 1000.00 | 500.00 | 73.75% |
| Carbopol ® 71GNF | 300.00 | 150.00 | 22.12% |
| (=Carbomer | | | |
| homopolymer type A) | | | |
| Aerosil ®200 (silicon dioxide) | 6.00 | 3.00 | 0.44% |
| Talc | 41.00 | 20.50 | 3.02% |
| Magnesiumstearate | 9.00 | 4.50 | 0.66% |
| Total | 1356.00 | 678.00 | 100.00% |

### Preparation

The hypericum extract is mixed with Carbopol® 71G NF, Aerosil®200 and talc for 10 minutes in a tumbling mixer. After adding magnesium stearate, mixing is continued for another 5 minutes. Using a model KilianSP300 eccentric press, the finished mixture is pressed into oval tablets with a breaking strength of 180 N.

### Active Substance Release

The active substance release from the tablet was established in a buffer of pH 7.2 over a period of 10 hours. The released amount of hypericin was measured.

| | Active substance |
|---|---|
| Time [h] | release [%] |
| 0 | 0 |
| 1 | 3.3 |
| 2 | 5.1 |
| 3 | 8.8 |
| 4 | 14.8 |
| 5 | 22.7 |
| 6 | 31.8 |
| 7 | 37.2 |
| 8 | 39.5 |
| 9 | 40.6 |
| 10 | 40.8 |

As the table shows, the active substance has been released only inadequately even after 10 hours (ca. 40%).

### COMPARISON EXAMPLE 3

### Preparation of a Composition According to the Invention Containing Hypericum Extract and Methocel®, a Water-Soluble Polymeric Matrix-Forming Agent

### Composition

| | Batch | | |
|---|---|---|---|
| | quantity | Quantity per | |
| Substance | [g] | tablet [mg] | Content % |
| Hypericum extract | 750.00 | 500.00 | 69.44% |
| Methocel ®K100MCR | 283.05 | 188.70 | 26.21% |
| (=hydroxymethylpropylcellulose) | | | |
| Aerosil ®200 (silicon dioxide) | 10.80 | 7.20 | 1.00% |
| Talc | 30.75 | 20.50 | 2.85% |
| Magnesium stearate | 5.40 | 3.60 | 0.50% |
| Total | 1080.00 | 720.00 | 100.00% |

### Preparation

The hypericum extract is mixed with Methocel®K100MCR, Aerosil®200 and talc for 10 minutes in a tumbling mixer. After adding magnesium stearate, mixing is continued for another 5 minutes. Using a model KilianSP300 eccentric press, the finished mixture is pressed into oval tablets with a breaking strength of 200 N.

### Active Substance Release

The active substance release from the tablet was established in a buffer of pH 7.2 over a period of 10 hours. The released amount of hypericin was measured.

| | Active substance |
|---|---|
| Time [h] | release [%] |
| 0 | 0 |
| 1 | 3.1 |
| 2 | 32.2 |
| 3 | 84.1 |
| 4 | 95.8 |
| 5 | 99.0 |
| 6 | 100.1 |
| 7 | 101.0 |
| 8 | 100.8 |
| 9 | 100.5 |
| 10 | 101.1 |

As the table shows, 80% of the active substance has been released after 3 hours.

### EXAMPLE 1

### Preparation of a Composition According to the Invention Containing Hypericum Extract and Kollidon® SR and Methocel® K4 M CR®, a Mixture of a Water-Soluble and a Water-Insoluble Polymeric Matrix-Forming Agent

### Composition

| | Batch | | |
|---|---|---|---|
| | quantity | Quantity per | |
| Substance | [g] | tablet [mg] | Content % |
| Hypericum extract | 1000.00 | 500.00 | 65.45% |
| Kollidon ®SR (=polyvinylacetate/ | 226.40 | 113.20 | 14.82% |
| polyvinylpyrrolidone blend) | | | |
| Methocel ® K 4 M CR | 204.20 | 102.10 | 13.36% |
| (=hydroxymethylpropylcellulose) | | | |
| Mannitol | 37.40 | 18.70 | 2.45% |
| Aerosil ® 200 (silicon dioxide) | 9.40 | 4.70 | 0.62% |
| Talc | 40.00 | 20.00 | 2.62% |
| Magnesium stearate | 10.60 | 5.30 | 0.69% |
| Total | 1528.00 | 764.00 | 100.00% |

### Preparation

The hypericum extract is mixed with Kollidon® SR, Methocel® K4 M CR, mannitol and talc for 10 minutes in a tumbling mixer. After adding magnesium stearate, mixing is continued for another 5 minutes. Using a model KilianSP300 eccentric press, the finished mixture is pressed into oval tablets with a breaking strength of 220 N.

### Active Substance Release

The active substance release from the tablet was established in a buffer of pH 7.2 over a period of 12 hours. The released amount of hypericin was measured.

| | Active substance |
|---|---|
| Time [h] | release [%] |
| 0 | 0 |
| 1 | 9.0 |
| 2 | 14.9 |
| 3 | 22.4 |
| 4 | 34.9 |
| 5 | 46.4 |
| 6 | 55.1 |
| 8 | 71.0 |
| 10 | 81.7 |
| 11 | 85.9 |
| 12 | 89.6 |

As the table shows, 81.7% of the active substance has been released after 10 hours.

### EXAMPLE 2

### Preparation of a Composition According to the Invention Containing Hypericum Extract and Kollidon® SR and Methocel® K 15 M CR®, a Mixture of a Water-Soluble and a Water-Insoluble Polymeric Matrix-Forming Agent

### Composition

| | Batch | | |
|---|---|---|---|
| | quantity | Quantity per | |
| Substance | [g] | tablet [mg] | Content % |
| Hypericum extract | 10000.00 | 500.00 | 65.45% |
| Kollidon ®SR (=polyvinylacetate/ | 2460.00 | 123.00 | 16.10% |
| polyvinylpyrrolidone blend) | | | |
| Methocel ® K 15 MCR | 2220.00 | 111.00 | 14.53% |
| (=hydroxymethylpropylcellulose) | | | |
| Aerosil ®200 (silicon dioxide) | 94.00 | 4.70 | 0.62% |
| Talc | 400.00 | 20.00 | 2.62% |
| Magnesium stearate | 106.00 | 5.30 | 0.67% |
| Total | 15280.00 | 764.00 | 100.00% |

### Preparation

The hypericum extract is mixed with Kollidon® SR, Methocel® K 15 M CR, Aerosil® 200 and talc for 10 minutes in a tumbling mixer, then compacted on a compactor (roller compactor by Alexanderwerk) applying a force of 5 kN/cm. The compacted substance is broken up, 1.0 mm, then mixed with magnesium stearate for 5 minutes. The finished mixture is pressed on a rotary pelleting machine of the model Korsch XL 100 into oval tablets with a breaking resistance of 150-160 N.

### Active Substance Release

The active substance release from the tablet was established in a buffer of pH 7.2 over a period of 12 hours. The released amount of hypericin was measured.

| | Active substance |
|---|---|
| Time [h] | release [%] |
| 0 | 0 |
| 1 | 12.8 |
| 2 | 22.1 |
| 3 | 31.8 |
| 4 | 41.9 |
| 5 | 55.5 |
| 6 | 67.6 |
| 7 | 79.9 |
| 8 | 92.0 |
| 9 | 98.8 |
| 10 | 100.1 |

As the table shows, 92.0% of the active substance has been released after 8 hours.

### EXAMPLE 3

### Preparation of a Composition According to the Invention Containing Gingko Biloba Extract, Kollidon® SR and Methocel® K 4 M CR®, a Mixture of a Water-Soluble and a Water-Insoluble Polymeric Matrix-Forming Agent

### Composition

| | Batch | | |
|---|---|---|---|
| | quantity | Quantity per | |
| Substance | [g] | tablet [mg] | Content % |
| Ginkgo biloba extract | 600.00 | 240.00 | 55.81% |
| Kollidon ®SR (=polyvinylacetate/ | 207.50 | 83.00 | 19.30% |
| polyvinylpyrrolidone blend) | | | |
| Methocel ® K 4 M CR | 200.00 | 80.00 | 18.60% |
| (=hydroxymethylpropylcellulose) | | | |
| Mannite | 25.00 | 10.00 | 2.33% |
| Aerosil ®200 (silicon dioxide) | 7.50 | 3.00 | 0.70% |
| Talc | 25.00 | 10.00 | 2.33% |
| Magnesium stearate | 10.00 | 4.00 | 0.93% |
| Total | 1075.00 | 430.00 | 100.00% |

### Preparation

The Ginkgo biloba extract is mixed with Kollidon® SR, Methocel® K 4 M CR, mannite, Aerosil® 200 and talc for 10 minutes in a tumbling mixer. After adding magnesium stearate, mixing is continued for another 5 minutes. Using a model KilianSP300 eccentric press, the finished mixture is pressed into oval tablets with a breaking strength of 150 N.

### Active Substance Release

The active substance release from the tablet was established in a buffer of pH 7.2 over a period of 12 hours.

| | Active substance |
|---|---|
| Time [h] | release [%] |
| 0 | 0 |
| 1 | 3.5 |
| 2 | 11.0 |
| 3 | 17.8 |
| 4 | 25.5 |
| 5 | 33.2 |
| 6 | 43.5 |
| 8 | 58.4 |
| 10 | 77.0 |
| 11 | 89.6 |
| 12 | 95.7 |

As the table shows, 80.0% of the active substance has been released after ca. 10 hours.

### EXAMPLE 4

### Preparation of a Composition According to the Invention Containing Gingko Biloba Extract, Kollidon® SR and Methocel® K 100 M CR, a Mixture of a Water-Soluble and a Water-Insoluble Polymeric Matrix-Forming Agent

### Composition

| | Batch | Quantity | |
|---|---|---|---|
| | quantity | pertablet | |
| Substance | [g] | [mg] | Content % |
| Ginkgo Biloba extract | 1200.00 | 240.00 | 72.73% |
| Kollidon ® SR | 300.00 | 60.00 | 18.18% |
| (=polyvinylacetate/ | | | |
| polyvinylpyrrolidone blend) | | | |
| Methocel ® K 100 MCR | 100.00 | 20.00 | 6.06% |
| (=hydroxymethylpropylcellulose) | | | |
| Aerosil ®200 (silicon dioxide) | 15.00 | 3.00 | 0.91% |
| Talc | 20.00 | 4.00 | 1.21% |
| Magnesium stearate | 15.00 | 3.00 | 0.91% |
| Total | 1650.00 | 330.00 | 100.00% |

### Preparation

The Ginkgo biloba extract is mixed with Kollidon® SR, Methocel® K 100 M CR, Aerosil® 200 and talc for 10 minutes in a tumbling mixer. After adding magnesium stearate, mixing is continued for another 5 minutes. The finished mixture is pressed on a rotary pelleting machine of the model Korsch XL 100 into oval tablets with a breaking resistance of 120 N.

### Active Substance Release

The active substance release from the tablet was established in a buffer of pH 7.2 over a period of 12 hours.

| | Active substance |
|---|---|
| Time [h] | release [%] |
| 0 | 0 |
| 1 | 7.5 |
| 2 | 18.2 |
| 3 | 33.3 |
| 4 | 48.6 |
| 5 | 67.0 |
| 6 | 87.5 |
| 8 | 94.1 |
| 10 | 98.0 |
| 11 | 99.6 |
| 12 | 100.1 |

As the table shows, 87.5% of the active substance has been released after 6 hours.

1. A pharmaceutical composition in solid form for the oral administration of active plant substances with delayed release, comprising a plant preparation containing the pharmacologically efficacious substances and a mixture of a polymeric water-soluble and a polymeric water-insoluble matrix-forming agent.

2. The pharmaceutical composition according to claim 1, containing a plant extract or a combination of plant extracts as plant preparation.

3. The pharmaceutical composition according to claim 1, containing hypericum extract.

4. The pharmaceutical composition according to claim 1, containing Ginkgo biloba extract.

5. The pharmaceutical composition according to claim 1 containing a pore-forming agent.

6. The pharmaceutical composition according to claim 1 containing one or a plurality of polymeric water-soluble matrix-forming agents selected among hydroxypropylmethylcellulose, hydroxypropylcellulose, poloxamer and polyvinylpyrrolidone.

7. The pharmaceutical composition according to claim 1 containing one or a plurality of polymeric water-insoluble matrix-forming agents selected among ethylcellulose, vinylpyrrolidone/vinylacetate copolymer, polyvinylpyrrolidone/polyvinylacetate mixture, polyacrylic acid copolymer, polymers of acrylic acid with polyalkenylether, polymethacrylic acid ester copolymer and methacrylic acid ester copolymer.

8. The pharmaceutical composition according to claim 1 containing the plant preparation at a content share of 50%.

9. The pharmaceutical composition according to claim 1 containing the plant preparation at a content share of over 65%.

10. The pharmaceutical composition according to claim 1 in the administration form of a tablet.
